(19) United States
(12) Patent Application Publication
(10) Pub. No.: US 2004/0172028 A1
(43) Pub. Date: Sep. 2, 2004

(54) High tibial osteotomy device

(76) Inventor: Greogory James Roger, New South Wales (AU)

Correspondence Address: JACOBSON HOLMAN PLLC, 400 SEVENTH STREET N.W., SUITE 600, WASHINGTON, DC, 20004, US

(21) Appl. No.: 10/479,628

(22) PCT Filed: Jun. 4, 2002
(86) PCT No.: PCT/AU02/00717

(30) Foreign Application Priority Data
Jun. 5, 2001 (AU) PR 5466

(51) Int. Cl. A61B 17/58

(52) U.S. Cl. 606/69

(57) ABSTRACT

A surgical device comprising a plate member (10) for mounting to a bone of a subject. The plate member (10) has a first end, a second end, a first face and a second face. At least one spacer member (20) is adapted to be mountable to at least a portion of the plate member (10) and relatively moveable towards and/or away from its first end (13). The spacer member (20) extends outwardly from the second face of the plate member (10) and is prevented from moving away from the first end (13) by a stop member (28) positioned between the first and second ends of the plate member (10).

### FIELD OF THE INVENTION

The present invention relates to a method and an apparatus for surgical reconstruction and intervention in orthopaedic surgery. The invention further relates to preferred constructions of surgical procedures especially high tibial osteotomies, femoral osteotomies, mandibular reconstruction, skull reconstruction and other such orthopaedic procedures.

### BACKGROUND ART

Correction of skeletal deformities and abnormalities, both inherent and from trauma can be performed using surgical procedures and devices. Distraction techniques whereby bone, which is of the incorrect length and/or alignment, is drawn out to the correct length are often used in conjunction with surgical devices.

In the case of reconstruction of skull bone, techniques and devices exist whereby the use of wires and scaffolding provides a reinforced structure to which bone can grow through and over.

High tibial osteotomy is a surgical procedure that is commonly used in the correction of “bowed legs” and in some circumstances “knock knees”. In the event of either of these disorders, abnormal load distribution and transfer is generated in and across the tibio-femoral joint. Abnormal load transfer causes increased load distribution on the medial tibial and femoral condyles in the case of “bow legs”, resulting in increased stresses, wear and patient pain. Increased loading on the medial condyles has a positive feedback effect on wear, resulting in exacerbated wear unless there is surgical intervention.

Conventionally, patients with such conditions are treated by several surgical techniques. Aged patients are usually treated with a total knee replacement, or in latter years with a Unicompartmental Knee replacement, when the medial articular surfaces of the knee are worn and the patient is suffering pain and lack of mobility. Younger subjects are more often treated by use of a high tibial osteotomy. The preferred approach for this procedure is a lateral approach of the proximal tibia. The procedure involves either removing a wedge of bone (referred to as a closing wedge) or by making a dome shaped cut, rotating the tibia and reducing the tibial plateau to the tibia. In either of the above procedures, the reduced “fracture” requires fixation such that fracture reunion can occur. Fixation is most commonly achieved by the use of plaster, staples, staple plates, fracture plates, fracture hardware and screws.

Another approach for the procedure is an approach from the worn side of the tibia, often the medial side, and to provide an opening wedge. In this case, a transverse cut is made across the tibia and the worn side is elevated such that the tibial plateau is more favourably aligned.

There are a number of complications associated with traditional high tibial osteotomy. The aim of the procedure is to realign the tibia with an over correction of typically about 3° such that, in the case of “bowed legs”, the loading is transferred from the medial side of the joint to the lateral side of the joint, allowing the medial side of the joint to recover.

Intraoperatively, it is difficult to effect the correct angle of adjustment. In the closing wedge procedure, a wedge of the correct thickness must be removed from the tibia. It has been found that if the second cut is not such that the correct size of wedge is removed, a third cut is a difficult action for correction. In the case of the domed osteotomy, the precise angle of shift for the tibia is difficult to determine accurately and difficult to adequately fix. Clinically it has been found that it is difficult to reliably to set the angle within 2° of the required angle. It has been found clinically that the opening wedge procedure is an easier procedure in so far as determining the correct angle, however, this procedure has substantial difficulties as will be discussed below.

Successful fixation of the surgically created fracture is a problem faced in respect of all known methods of high tibial osteotomy. The wedge is constantly being forced closed by muscle, ligament and weight forces. To overcome this problem, bone harvested from other parts of the patient has been used to hold open the gap. Also plate and screw hardware has been used to hold open the gap created by the removal of the wedge. These methods have often failed due to collapse of the bone autograft prior to osteo-integration and bone union, and from failure of the plate/screw system. The plate/screw system offers limited resistance to torsional loads and axial rotation. This often results in screw pull-out or screw failure due to shear.

In the case of surgical intervention on the lateral aspect, there are several complications including the anatomical location of a major nerve in the proposed site for the osteotomy, and that once the osteotomy has been performed, total knee replacement, should that option be required at a later date, is complicated. In addition, many incisions in this area pose increased risk of limiting vascularity, resulting in tissue failure. Also, a lateral approach requires shortening of the fibula and as well there is a reduction in overall leg length.

Secondary alignment is often difficult to control with known methods of high tibial osteotomy. There should be no inadvertent posterior tilt or rotation. Such misalignments result in subsequent deformities and complications. The closing wedge procedure poses alignment problems as two separate cuts must be made, such that upon loading the faces of the cuts meet at the cortex and the cortex acts as a stabilising means for the procedure. There is not a uniform meeting of the proximal and distal cortices at the osteotomy site, leading to increased stress concentration and deformity.

In order to overcome such complications, Puddu (U.S. Pat. No. 5,749,875) uses a system of plates and screws of varying sizes and geometries and a calibrated wedge tool for opening a resected tibial wedge to determine the size of the plate required. Again, excessive loads to screws and plates across osteotomy sites has resulted in failure due to the heads of the screws shearing off under load. Such failure results in further complications and restorative surgery.

The present invention is directed to providing an alternative to the existing techniques and apparatus for use in the performance of surgical procedures, especially those requiring reconstructive orthopaedic intervention, such as high tibial osteotomies and mandibular or other skull bone reconstructions. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### DISCLOSURE OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention is a surgical device comprising:

a plate member that is mountable to a bone of a subject, the plate member having a first end, a second end, a first face, a second face, and a stop means between said first end and said second end; and

at least one spacer member mountable to at least a portion of the plate member and relatively moveable towards and/or away from the first end, the spacer member, on mounting, extending outwardly from the second face of the plate member;

wherein on abutment of the spacer member with the stop means, the spacer member is prevented from undergoing further movement in a direction away from the first end of the plate member.

In one embodiment, the plate member further comprises a receiving means for a bone fixation device, the receiving means having an abutment surface wherein on engagement of a fixation device fixed in a bone with the abutment surface and relative movement between the plate member and the bone thereto leads to an alteration in the stress within the fixation device, the altered stress within the fixation member being such that further relative movement between the plate member and the bone is reduced.

In another embodiment of the first aspect, the plate member has a first side and a second side. The at least one abutment surface can extend from the first side of the plate member to the second side of the plate member on at least a portion of the first face of the plate member. The at least one abutment surface can have a first side and a second side. Examples of fixation devices that can engage with the at least one abutment surface include bone screws and circumferential fixation devices including cable and block fasteners, screw-driven circumferential clamps and wedge/block mechanisms.

In another embodiment of the first aspect, the plate member can have an orifice extending through the at least one abutment surface. The orifice can extend through the plate member and through the second face of the plate member. In one embodiment, the orifice can be ovoid or elongate. In another embodiment, the fixation device can have a first end to engage with the abutment surface of the plate member, a second end to engage with the subject's bone and an intermediate portion between the first and second ends which can pass through the ovoid or elongate orifice of the plate member. Examples of fixation devices that can engage with the at least one abutment surface and engage with the bone include screws, bolts, nails and pins.

In a second aspect, the present invention is a surgical device comprising:

a plate member that is mountable to a bone of a subject, the plate member having a first end, a second end, a first face and a second face; and

at least one spacer member extending outwardly from the second face of the plate member and positionable between two bone surfaces formed in the bone of the subject;

wherein the plate member has at least one abutment surface engageable with a fixation device used to affix the plate member to the bone, such that relative movement between the plate member and the bone induces an altered stress within the fixation device, the altered stress within the fixation device being such that further relative movement between the plate member and the bone is substantially prevented or at least reduced.

In a first embodiment of the second aspect, the spacer member can be formed integrally with the plate member.

In an embodiment of the first and second aspects, the at least one abutment surface is at an angle of inclination to the first face of the plate member. The inclination of the abutment surface can be upwardly from the first end of the plate member toward the second end of the plate member, or from the second end of the plate member to the first end of the plate member. In a further embodiment, the plate member can have a first side and a second side. The at least one abutment surface can extend from the first side of the plate member to the second side of the plate member on at least a portion of the first face of the plate member. The at least one abutment surface can itself have a first side and a second side. The at least one abutment surface can extend outwardly from the first surface of the plate.

In another embodiment of the first and second aspects, the abutment surface can be recessed within the body of the plate member. Examples of fixation devices that can engage with the at least one abutment surface include bone screws, and circumferential fixation devices including cable and block fasteners, screw-driven circumferential clamps and wedge/block mechanisms.

In another embodiment of the second aspect, the plate member can have an ovoid or elongate orifice extending from the abutment surface through the plate member, to the second face of the plate member. The ovoid or elongate orifice preferably extends along at least a portion of the abutment surface. The ovoid or elongate orifice can have a short axis and a long axis. The long axis can be in alignment with the longitudinal axis extending between a first end of the plate member and a second end of the plate member.

In the second aspect, a fixation device can have a first end to engage with the abutment surface of the plate member, a second end to engage with the subject's bone and an intermediate portion which can pass through the orifice of the plate member, can be used to affix the plate member to the bone. Examples of fixation devices that can engage with the at least one abutment surface and engage with the bone include screws, bolts, nails and pins.

In yet another embodiment of the first and second aspects, the plate member preferably has at least one abutment surface between the first end of the plate member and the at least one spacer member, or between the second end of the plate member and the at least one spacer member. More preferably, the plate member has at least one abutment surface between the first end of the plate member and the at least one spacer member and at least one abutment surface between the second end of the plate member and the at least one spacer member. Still more preferably, the plate member has a plurality of abutment surfaces between the second end of the plate member and the at least one spacer member and at least one abutment surface between the first end of the plate member and the at least one spacer member. More preferably still, at least one of the abutment surfaces can have an orifice extending from the abutment surface through the plate member, to the second face of the plate member. The at least one orifice can be circular or ovoid or elongate.

In a further embodiment of the first and second aspects, the plate member and the at least one spacer member can be made of a biocompatible material, preferably of a biocompatible metal or metal alloy. Examples of metals and metal alloys include stainless steel, titanium, titanium alloys and cobalt-chrome alloys. In another embodiment, the at least one spacer member and/or the plate member can be formed from a biologically active material. For example, the spacer member can be formed from a block of calcium phosphate.

In a still further embodiment of the first and second aspects, the spacer member can include a built-in substance delivery system. The built-in delivery system can be used for the delivery of at least one drug and/or therapeutic agent to a site within the patient's body. Examples of drugs or therapeutic agents include prophylactic antibiotic or steroids, hydroxyapatite, calcium phosphate, bone morphogenic proteins and growth factors.

In a preferred embodiment of the second aspect, the present invention has a plate member having at least one spacer member mountable to at least a first portion of the plate member, the spacer member being relatively moveable to the plate member from the first end of the plate member toward and away from the first end of the plate member. The plate member has a stop means for preventing the further relative motion of the spacer member toward the second end of the plate member. The plate member has at least one abutment surface between the stop means and the first end of the plate member, and at least one abutment surface between the stop means and the second end of the plate member. The respective abutment surfaces are recessed below the first surface of the plate member. Ovoid or elongate orifices extend through the plate member, passing through the at least one abutment surfaces. The ovoid or elongate orifices can be co-linear or offset.

In a third aspect, the present invention is a method of carrying out an osteotomy on a bone of a patient to align the bone or correct a deformity comprising the steps of:

(a) forming a saw cut in the bone of a patient;

(b) causing the cut to be widened on at least one side so as to align or correct the deformity of the bone;

(c) placing the at least one spacer member of the surgical device according to the first aspect within the widened cut such that the at least one spacer member is in contact with cortical bone on each side of the cut; and

(d) securing the plate member to the bone of the patient.

In an embodiment of the third aspect, the method can comprise a further step of engaging the plate member to the at least one spacer member. Preferably, the further step of engaging the plate member with the at least one spacer member is performed between step (c) and step (d).

In a preferred embodiment of the third aspect, the present invention relates to a process for carrying out a high tibial osteotomy, where the saw cut is performed through a tibia of a patient substantially in the medio-lateral plane adjacent to the head of the fibula.

Many combinations of fixation devices can be used to secure the plate member of the devices of the first, second and third aspects to the bone of a subject. Such fixation devices include screws, bolts, nails, pins, screw driven circumferential clamps, cable and block fasteners and a combination of one or more of these devices.

### BRIEF DESCRIPTION OF THE DRAWINGS

By way of example only, preferred embodiments of the invention are described with reference to the accompanying drawings, in which:

FIG. 1 is a perspective view of one embodiment of a plate member according to the present invention having two protruding abutment surfaces;

FIG. 2 is a perspective view of another embodiment of a plate member according to the present invention having two recessed abutment surfaces;

FIG. 3 is a perspective view of another embodiment of a plate member according to the present invention having a protruding abutment surface and an elongate orifice;

FIG. 4 is a perspective view of another embodiment of a plate member according to the present invention having a recessed abutment surface and an elongate orifice;

FIG. 5 is a perspective view of another embodiment of a plate member according to the present invention having a recessed abutment surface and an elongate orifice;

FIG. 6 is a longitudinal cross sectional view of the plate member of FIG. 5;

FIG. 7 is a perspective view of another embodiment of a plate member according to the present invention having a protruding abutment surface and an elongate orifice;

FIGS. 8a and 8b are cross-sectional and perspective views of a plate member having a recessed and chamfered abutment surface for engagement with the head of a bone screw, respectively;

FIG. 9 is a cross sectional view of one embodiment of a plate member and spacer member in situ in bone of a subject;

FIGS. 10(a), 10(b) and 10(c) are views of another embodiment of a plate member and spacer member according to the present invention; and

FIG. 11 is a perspective view of the plate member and spacer member of FIG. 10 in an assembled configuration.

FIG. 12 is a perspective view of the plate member mounted to the bone of a subject by fixation means.

### DESCRIPTION OF THE INVENTION

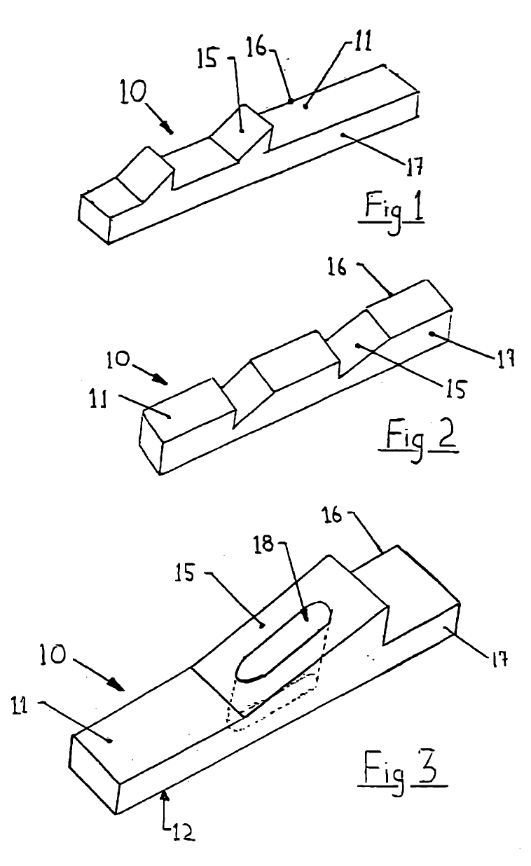

As an example only, a configuration of the abutment surface 15 of a plate member for one embodiment of a surgical device according to the present invention is depicted in FIG. 1. In this example, the abutment surface 15 extends upwardly from the first face 11 of the plate member 10, and extends across the width of the first face of the plate member from a first side 16 of the plate member 10 to a second side 17 of the plate member 10. Circumferential fixation devices including cable and block fasteners, screw-driven circumferential clamps and wedge/block mechanisms can be used to engage with the abutment surface 15 to secure the depicted plate member 10 to the bone of a subject.

FIG. 2 depicts an example of the abutment surface 15 when recessed within the plate member 10, and extending across the width of first face 11 of the plate member 10 from the first side 16 to the second side 17. Circumferential fixation devices including cable and block fasteners, screw-driven circumferential clamps and wedge/block mechanisms can again be used to engage with the abutment surface 15 to secure the plate member 10 to the bone of a subject.

A further example of a protruding abutment surface 15 of a plate member 10 is depicted in FIG. 3. The abutment surface 15 extends across the width of the first face of the plate member 11 from the first side 16 of the plate member 10 to the second side 17 of the plate member 10. An elongate orifice 18 passes through the abutment surface 15, through the plate member 10 and out through the second face 12 of the plate member 10. Fixation devices that can engage with the abutment surface 15 of this example and secure the plate member 10 to the bone of a patient include screws, bolts, nails, pins, screw-driven circumferential clamps, cable and block fasteners and a combination of one or more of these devices.

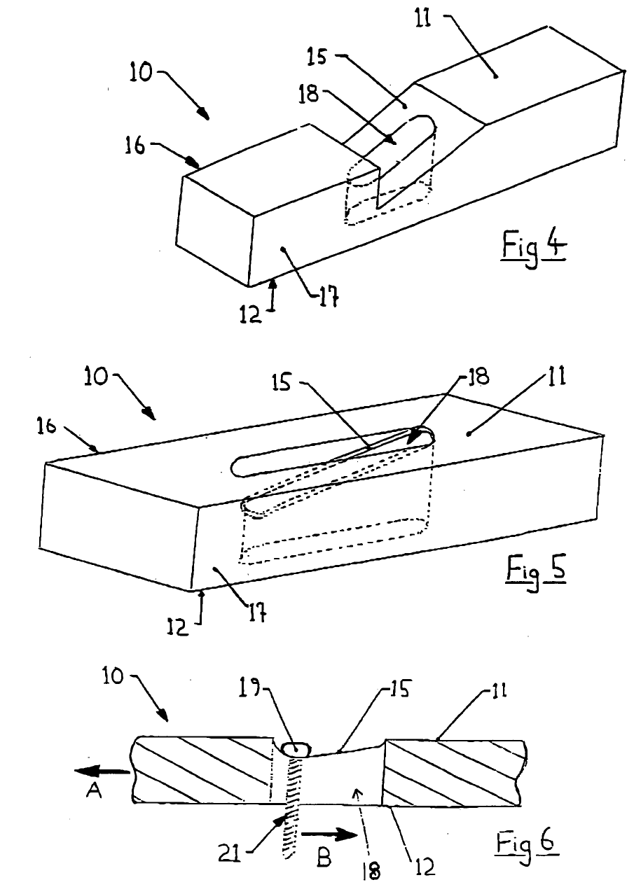

FIG. 4 depicts an example of a plate member 10 having a recessed abutment surface 15 extending across the width of the first face of the plate member 11 from the first side 16 of the plate member 10 to the second side 17 of the plate member 10. An elongate orifice 18 passes through the abutment surface 15, through the plate member 10 and out through the second face 12 of the plate member 10. Fixation devices that can engage with the abutment surface 15 of this example and secure the plate member 10 to the bone of a subject include screws, bolts, nails, pins, screw driven circumferential clamps, cable and block fasteners and a combination of one or more of these devices.

FIG. 5 depicts an example of the abutment surface 15 recessed within the plate member 10. In this embodiment, the abutment surface extends across only a portion of the width of the first face 11 of the plate member 10. An elongate orifice 18 passes through the abutment surface 15, through the plate member 10 and out through the second face 12 of the plate member 10. Fixation devices that can engage with the abutment surface 15 of this example and secure the plate member 10 to the bone of a patient include screws, bolts, nails and pins.

FIG. 6 depicts a longitudinal cross sectional view of a portion of the plate 10 of FIG. 5 through the elongate orifice 18. The head 19 of a screw 21 engages with the abutment surface 15 to secure the plate member 10 to the bone of a subject. The gradient of the abutment surface 15 is such that relative motion between the plate member in the direction A and the screw member 21 in the direction of B, is opposed due to the abutment surface increasing the stress in the screw member 17 by virtue of a wedge-type action.

Where there a plurality of abutment surfaces 15, stress due to relative motion can be distributed between a plurality of fastening means.

The gradient of the abutment surface 15 is such that as the head of the screw member 17, in the event of relative motion along the abutment surface in the direction of B, does not fail due to increased stress.

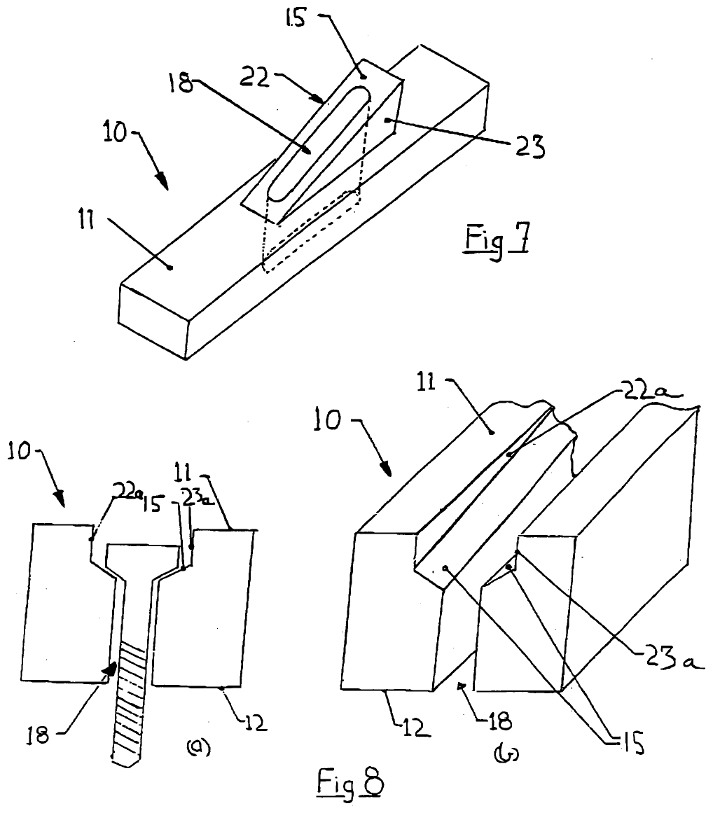

FIG. 7 depicts a further example of a plate member 10 where the abutment surface 15 is protruding from the first face 11. In this embodiment, the abutment surface 15 extends across a portion of the width of the first face of the plate member 10 and has a first face 22 and a second face 23. An elongate orifice 18 passes through the abutment surface 15, through the plate member 10 and out through the second face 12 of the plate member 10. Fixation devices that can engage with the abutment surface 15 of this example and secure the plate member 10 to the bone of a subject include screws, bolts, nails, pins, screw driven circumferential clamps, cable and block fasteners and a combination of one or more of these devices.

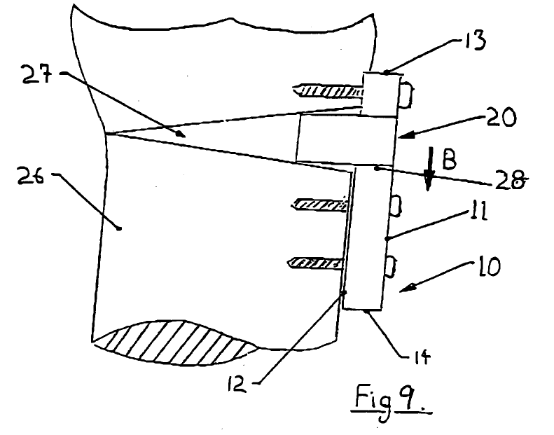

FIGS. 8(a) and 8(b) depict an example of a plate member 10 where the abutment surface 15 is recessed within the plate member 10. The abutment surface is chamfered such that a chamfered or substantially hemispherical head of a screw can engage with the abutment surface 15. A transverse cross section of the plate member is shown in FIG. 8(a). The abutment surface has a first face 22a and a second face 23a, however as the abutment surface is recessed in FIG. 8, the first face 22a and the second face 23a are internal the plate member 10. The first face 22a and the second face 23a of the abutment surface extend downwardly from the first face 11 of the plate member 10 toward the second face 12 of the plate member 10. At the termination of the first face 22a and the second face 23a of the abutment surface, the respective portions of the abutment surface 15 slope downwardly and toward each other. An elongate orifice 18 passes through the abutment surface 15, through the plate member 10 and out through the second face 12 of the plate member 10. Fixation devices that can engage with the abutment surface 15 of this example and secure the plate member 10 to the bone of a patient include screws, bolts, nails and pins. FIG. 8(b) shows that portion of the plate member having the chamfered abutment surface 15 with an elongate orifice passing through the plate member and out through the second face 12 of the plate member 10.

FIG. 9 depicts a preferred embodiment of the device according to the present invention in use. The invention is depicted being used in a tibial osteotomy. In this arrangement, the spacer member 20 is depicted positioned within the opening of an osteotomy 27. The plate member 10 is engaged with the spacer member 20 in a manner such that the spacer member is stopped from moving from the first end 13 of the plate member 10 toward the second end of the plate member by a stop member 28 incorporated within the plate member. In FIG. 9, the abutment surfaces are recessed within the plate member. There is also only one abutment surface between the stop member of the plate member and the first end of the plate member, and at least one abutment surface between the stop member and the second end of the plate member. The abutment surfaces extend across a portion of the first face 11 of the plate member 10. An elongate orifice passes through the abutment surface, through the plate member 10 and out through the second face 12 of the plate member 10. The abutment surfaces are inclined such that relative motion of the plate member 10 in the direction of B is reduced or inhibited. Fixation devices that can be used to secure the plate member to the bone of the patient include screws, bolts, nails and pins.

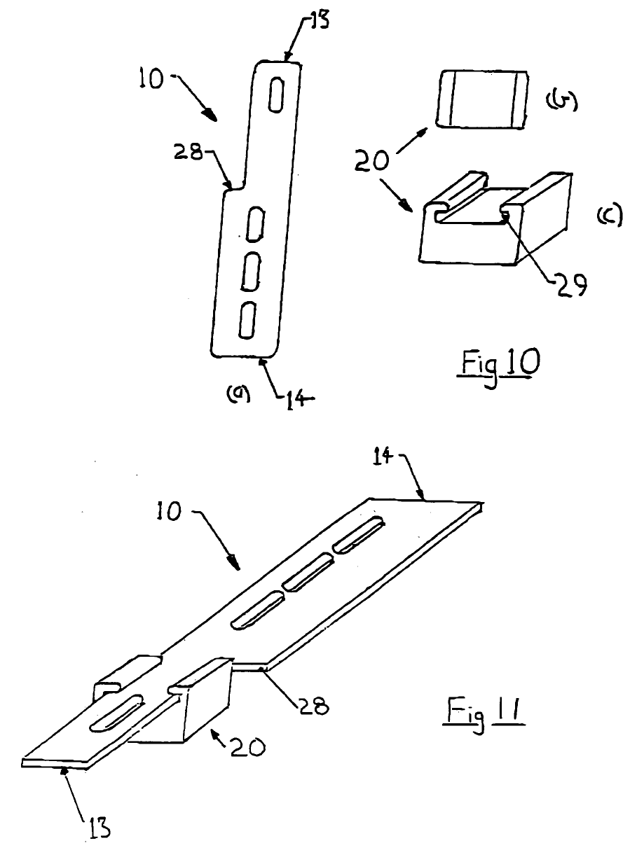

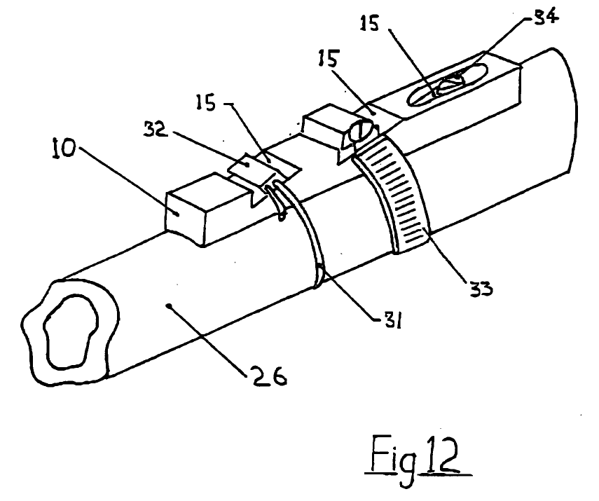

FIGS. 10(a), 10(b) and 10(c) depict one embodiment of the plate member 10 and spacer member 20. FIG. 10(a) depicts the plate member 10 having a stop member 28 that is adapted to prevent relative sliding motion between the plate member 10 and the spacer member 20 in a direction away from the first end 13 once the spacer member 20 has come into abutment with the stop member 28. In this example, the stop member 28 is a thickening or widening of the plate member. FIG. 10(b) depicts the face of the spacer member 20 that slides against the second face of the plate member 10. FIG. 10(c) depicts an undercut 29 in the spacer member 20 through which the plate member 10 is adapted to be slidable.

FIG. 11 depicts the plate member 10 and spacer member 20 of FIG. 10 in an assembled configuration. The spacer member 20 can slide from the first end 13 of the plate member 10 toward the second end of the plate member until it comes into abutment with the stop member 28 of the plate member. In this example, the stop member 28 consists of a widening of the plate member 10.

FIG. 12 depicts an example of the plate member 10 fixed to a bone 26 using several different types of fixation devices engaging with the abutment surface 15 of the plate member 10. A cable 31 and block fastener 32 is shown as one example of fixation means suitable for fixing the plate member 10 to the bone 26. Another example of a fixation means is a screw-driven clamp means 33. A further example of a fixation means suitable for fixing the plate member 10 to the bone 26 is a bone screw 34. Although not depicted in this figure, many further combinations of fixation devices can be used to secure the plate member of the devices of the first, second and third aspects to the bone of a subject. Such fixation devices include screws, bolts, nails, pins, screw driven circumferential clamps, cable and block fasteners and a combination of one or more of these devices.

It will be appreciated that the first end 13 of the depicted plate member 10 could be mounted to the bone of a subject so as to face proximally or distally as circumstances dictate.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A surgical device comprising:
a plate member that is mountable to a bone of a subject, the plate member having a first end, a second end, a first face, a second face, and a stop means between said first end and said second end; and
at least one spacer member mountable to at least a portion of the plate member and relatively moveable towards and/or away from the first end, the at least one spacer member, on mounting, extending outwardly from the second face of the plate member;
wherein on abutment of the spacer member with the stop means, the spacer member is prevented from undergoing further movement in a direction away from the first end of the plate member.

2. A surgical device of claim 1 wherein the plate member further comprises at least one receiving means for a bone fixation device, the receiving means having an abutment surface wherein on engagement of a fixation device fixed in a bone with the abutment surface and relative movement between the plate member and the bone thereto leads to an alteration in the stress within the fixation device, the altered stress within the fixation member being such that further relative movement between the plate member and the bone is reduced.

3. A surgical device of claim 2 wherein the receiving means has a first side and a second side.

4. A surgical device of claim 2 or claim 3 wherein the abutment surface is recessed within the body of the plate member.

5. A surgical device of any one of claims 2 to 4 wherein the plate member has at least one abutment surface between the first end of the plate member and the stop means.

6. A surgical device of any one of claims 2 to 5 wherein the plate member has at least one abutment surface between the first end of the plate member and the stop means and at least one abutment surface between the second end of the plate member and the stop means.

7. A surgical device of any one of claims 2 to 6 wherein the at least one abutment surface is at an inclination to the first face of the plate member.

8. A surgical device of any one of claims 2 to 7 wherein the plate member has an orifice extending through at the at least one abutment surface, the plate member and the second face of the plate member.

9. A surgical device of claim 8 wherein the orifice in the plate member is elongate.

10. A surgical device of claim 8 or claim 9 wherein the fixation device is selected from the group comprising a bone screw, a screw-driven circumferential clamp, a cable, and a block fastener.

11. A surgical device of claim 10 wherein the fixation device has a first end for engagement with the at least one abutment surface of the plate member, a second end for engagement with the subject's bone and an intermediate portion between the first and second ends of the fixation device which can pass through the orifice in the plate member.

12. A surgical device of any one of the preceding claims wherein the plate member and the at least one spacer member are made from a biocompatible material.

13. A surgical device of claim 12 wherein the biocompatible material is selected from a group comprising a biocompatible metal, a biocompatible metal alloy, stainless steel, titanium, titanium alloys, and cobalt-chrome alloys.

14. A surgical device of claim 12 wherein the at least one spacer member and/or the plate member is made from a biologically active material.

15. A surgical device comprising:
a plate member that is mountable to a bone of a subject, the plate member having a first end, a second end, a first face and a second face; and
at least one spacer member extending outwardly from the second face of the plate member and positionable between two bone surfaces formed in the bone of the subject;
wherein the plate member has at least one abutment surface engageable with a fixation device used to affix the plate member to the bone, such that relative movement between the plate member and the bone induces an altered stress within the fixation device, the altered stress within the fixation device being such that further relative movement between the plate member and the bone is substantially prevented or at least reduced.

16. A surgical device of claim 15 wherein the spacer member is formed integrally with the plate member.

17. A surgical device of claim 15 wherein the abutment surface is recessed within the body of the plate member.

18. A surgical device of any one of claims 15 to 17 wherein the plate member has at least one abutment surface between the first end of the plate member and the at least one spacer member.

19. A surgical device of any one of claims 15 to 18 wherein the plate member has at least abutment surface between the first end of the plate member and the at least one spacer member and at least one abutment surface between the second end of the plate member and the at least one spacer member.

20. A surgical device of any one of claims 15 to 19 wherein the at least one abutment surface is at an angle of inclination to the first face of the plate member.

21. A surgical device of any one of claims 15 to 20 wherein the plate member has an orifice extending through at the at least one abutment surface, the plate member and the second face of the plate member.

22. A surgical device of claim 21 wherein the orifice in the plate member is elongate.

23. A surgical device of claim 21 or claim 22 wherein the fixation device is selected from the group comprising a bone screw, a screw-driven circumferential clamp, a cable and block fastener, and a combination of one or more of these devices.

24. A surgical device of claim 23 wherein the fixation device has a first end for engagement with the abutment surface of the plate member, a second end for engagement with the subject's bone and an intermediate portion between the first and second ends of the fixation device which can pass through the orifice in the plate member.

25. A surgical device of any one of claims 15 to 24 wherein the plate member and the at least one spacer member are made from a biocompatible material.

26. A method of carrying out an osteotomy on a bone of a patient to align the bone or correct a deformity comprising the steps of:
(a) forming a saw cut in the bone of a patient;
(b) causing the cut to be widened on at least one side so as to align or correct the deformity of the bone;
(c) placing the at least one spacer member of the device of any one of the preceding claims within the widened cut such that the at least one spacer member is in contact with cortical bone on each side of the cut; and
(d) securing the plate member to the bone of the patient with a bone fixation device.

27. The method according to claim 26, further comprising a step of engaging the plate member with the at least one spacer member.

28. The method according to claim 27, wherein the step of engaging the plate member with the at least one spacer member is performed between step (c) and step (d).

29. The method according to any one of claims 26 to 29, wherein the saw cut is performed through a tibia of a subject substantially in the medio-lateral plane adjacent to the head of the fibula.
